(19) United States
(12) Patent Application Publication
(10) Pub. No.: US 2024/0206993 A1
(43) Pub. Date: Jun. 27, 2024

(54) SYSTEM AND METHOD TO POSITION SURGICAL SCOPE IN ROBOTIC SYSTEM

(71) Applicant: Auris Health, Inc., Redwood City, CA (US)

(72) Inventors: Theodore Leclere, San Francisco, CA (US); Jalen L. Wize, San Francisco, CA (US); Charles J. Scheib, Loveland, OH (US)

(21) Appl. No.: 18/390,614

(22) Filed: Dec. 20, 2023

Related U.S. Application Data
(60) Provisional application No. 63/434,194, filed on Dec. 21, 2022.
(60) Provisional application No. 63/434,199, filed on Dec. 21, 2022.

(51) Int. Cl. A61B 34/30; A61B 1/00; A61B 1/008

(52) U.S. Cl. CPC A61B 34/30; A61B 1/00097; A61B 1/00149; A61B 1/00154; A61B 1/008; A61B 2034/301; A61B 2090/061

(57) ABSTRACT

A system includes a surgical scope, a feed device, and a controller. The surgical scope is configured to extend through a surgical opening in a body wall of a patient and into a body cavity and includes a distal tip having a lens configured to visualize an anatomical structure. The feed device is operable to selectively advance and retract the surgical scope relative to the body wall. The controller is in communication with the surgical scope and the feed device, and is configured to determine a present target distance measured from the distal tip to the anatomical structure, and compare the present target distance to a threshold target distance. Based on the comparison, the controller is configured to at least one of control the feed device to advance, retract, or halt the surgical scope longitudinally relative to the body wall, or provide a notification to a user of the system.

### PRIORITY

This application claims the benefit of U.S. Provisional Pat. App. No. 63/434,194, entitled “Insertion System for Surgical Scope in Robotic System,” filed Dec. 21, 2022, and to U.S. Provisional Pat. App. No. 63/434,199, entitled “System and Method to Position Surgical Scope in Robotic System,” filed Dec. 21, 2022, the disclosures of which are incorporated by reference herein, in their entireties.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first illustrative robotic system configured for a laparoscopic procedure;

FIG. 2 depicts a perspective view of a second illustrative robotic system;

FIG. 3 depicts an end elevational view of the robotic system of FIG. 2;

FIG. 4 depicts an end elevational view of the robotic system of FIG. 2 including an illustrative pair of robotic arms;

FIG. 5 depicts a partially exploded perspective view of the robotic arm of FIG. 4 having an instrument driver and an illustrative surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7 depicts a schematic view of an illustrative surgical scope and a pair of surgical instruments, showing a deflectable shaft portion of the surgical scope and distal ends of the surgical instruments inserted through a body wall and into a body cavity of a patient;

FIG. 8 depicts a perspective view of another illustrative robotic system having a robotic arm, a surgical scope having a deflectable distal shaft portion, and a cannula having an articulation feature;

FIG. 9 depicts a schematic side elevational view of the surgical scope and the cannula of FIG. 8;

FIG. 10 depicts a side elevational view of the cannula of FIG. 8, showing the articulation feature in an articulated state;

FIG. 11 depicts a side cross-sectional view of the cannula of FIG. 8 with the articulation feature in an articulated state;

FIG. 12A depicts a perspective view of the surgical scope and the cannula of FIG. 8 inserted into the body cavity of a patient, showing the articulation feature in a first articulated state and the deflectable distal shaft portion in a first deflected state;

FIG. 12B depicts a perspective view of the surgical scope and the cannula of FIG. 12A, showing the articulation feature in a second articulated state and the deflectable distal shaft portion in a second deflected state;

FIG. 12C depicts a perspective view of the surgical scope and the cannula of FIG. 12A, showing the deflectable distal shaft portion inserted further into the body cavity;

FIG. 12D depicts a perspective view of the surgical scope and the cannula of FIG. 12A, showing a distal end of the deflectable distal shaft portion in an articulated state;

FIG. 13A depicts a schematic side sectional view of an illustrative surgical scope system that includes a surgical scope, a cannula, and a scope feed device having an integrated motor operable to advance and retract the surgical scope and the sheath relative to the body wall of a patient, showing the surgical scope and sheath in a distally advanced position;

FIG. 13B depicts a schematic side sectional view of the surgical scope system of FIG. 13A, showing the surgical scope in a proximally retracted position;

FIG. 14A depicts a schematic side sectional view of the cannula of FIG. 13A in combination with an obturator after they have been inserted distally through the body wall;

FIG. 14B depicts a schematic side sectional view of the cannula and the obturator of FIG. 14A, showing the cannula and obturator having been retracted proximally such that a retention feature of the cannula contacts the body wall;

FIG. 14C depicts a schematic side sectional view of the surgical scope system of FIG. 13A, showing the cannula reconfigured after removal of the obturator of FIG. 14A

FIG. 14D depicts a schematic side sectional view of the surgical scope system of FIG. 13A, showing distal advancement of a cannula sheath by the scope feed device and articulation of a distal end of the cannula sheath;

FIG. 15 depicts a schematic side sectional view of another illustrative surgical scope system having a motor located remotely from a scope feed device body;

FIG. 16 depicts a side partial-sectional view of an illustrative robotic system having a scope feed device with a motor located remotely from the scope feed device body;

FIG. 17 depicts a side partial-sectional view of another illustrative robotic system having a scope feed device with a motor located remotely from the scope feed device body;

FIG. 18 depicts a schematic side sectional view of another illustrative surgical scope system having a scope feed device with a manually actuated drive member;

FIG. 19 depicts a perspective view of another illustrative scope feed device having a manually actuated drive member;

FIG. 20A depicts a side sectional view of the scope feed device of FIG. 19, taken along line 20-20 of FIG. 19, showing the scope feed device in an unlatched state relative to a surgical scope assembly;

FIG. 20B depicts another side sectional view of the scope feed device and surgical scope assembly of FIG. 20A, taken along line 20-20 of FIG. 19, showing the scope feed device in a latched state relative to the surgical scope assembly;

FIG. 20C depicts another side sectional view of the scope feed device and surgical scope assembly of FIG. 20A, taken along line 20-20 of FIG. 19, showing a drive member of the scope feed device having been actuated by a user-actuatable feature while in the latched state;

FIG. 21A depicts a top sectional view of the scope feed device and surgical scope assembly of FIGS. 19 and 20A, taken along line 21-21 of FIG. 19, showing the scope feed device in the unlatched state relative to the surgical scope assembly;

FIG. 21B depicts a top sectional view of the scope feed device and surgical scope assembly of FIG. 21A, taken along line 21-21 of FIG. 19, showing the scope feed device in the latched state relative to the surgical scope assembly;

FIG. 22 depicts a schematic side sectional view of another illustrative surgical scope system that includes a surgical scope, a cannula, a scope feed device, and an optical distance measurement system;

FIG. 23 depicts a diagrammatic view of an illustrative control system that incorporates the surgical scope system of FIG. 22; and

FIG. 24 depicts a diagrammatic view of an illustrative method of operating the surgical scope system of FIG. 22.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. Example of Robotically-Enabled Medical System

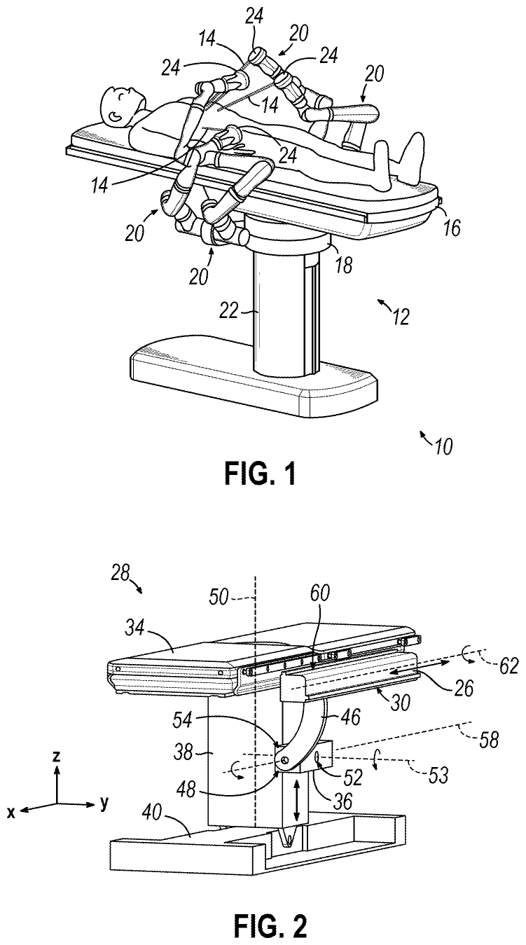

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

### A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

### B. Example of Robotic System with Bar Carriage

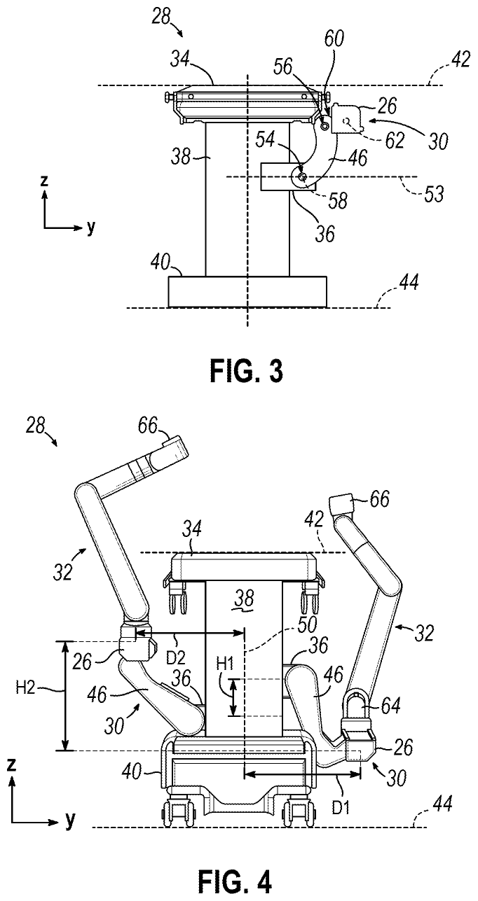

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

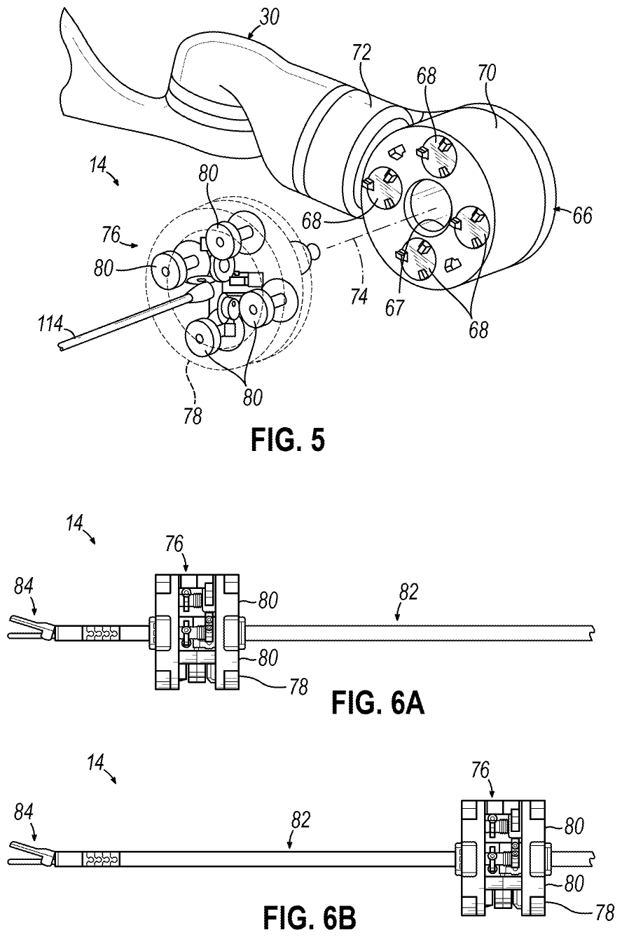

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

### C. Example of Surgical Instrument with Instrument-Based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### II. Surgical Scope Having Deflectable Shaft

In robotically assisted laparoscopic procedures, it may be desirable to mount a surgical instrument in the form of a surgical scope, also referred to as a surgical camera or a laparoscope, to a robotic arm of the robotic system to provide real-time visualization of a target surgical site and surrounding anatomical structures with the patient's body cavity during the procedure. Some conventional surgical scopes implemented in robotic systems may include an elongate rigid base and an elongate rigid shaft extending distally from the base. The surgical scope may be mounted to the head of a robotic arm, where the head is also docked directly to a cannula through which the scope shaft is inserted to access the body cavity. This configuration may tend to result in the surgical scope and its robotic arm consuming valuable space in the workspace located directly above the patient. This may ultimately tend to restrict the range of motion of the surgical scope and other robotically-controlled surgical instruments operating within this workspace, thereby limiting the reach and access of the surgical scope and the surgical instruments and risking collision between these devices and their respective robotic arms in the workspace. Accordingly, it may be desirable to provide alternative configurations of surgical scopes that promote greater reach and access of the surgical scope and accompanying surgical instruments of a robotic system.

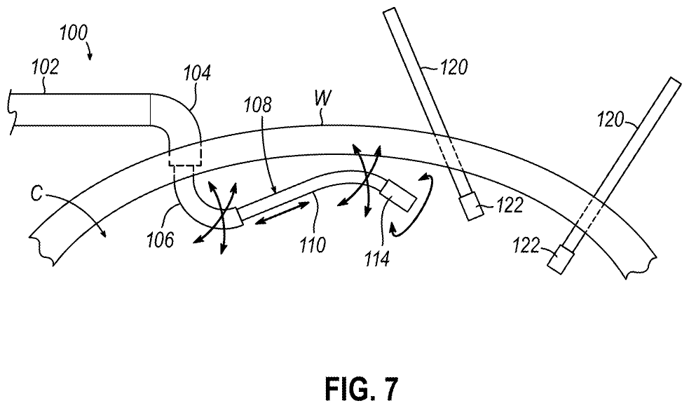

FIG. 7 shows an example of a surgical scope (100) that is constructed such that a portion of surgical scope (100) is non-rigid, thus enabling a base (not shown) of surgical scope (100) and its corresponding robotic arm (not shown) to be positioned remotely from the location at which the surgical scope enters through the body wall (W) and into a body cavity (C) of a patient, such as an abdominal cavity. Surgical scope (100) includes an elongate outer sheath (102) (also referred to as an “outer tube”), which may be rigid or deflectable; and a curved joint (104) at a distal end of outer sheath (102), which may also be rigid with a predefined curvature or deflectable and configured to assume a curved state as shown. As used herein, the term “deflectable” encompasses configurations that are configured to deflect relative to an initial axis, including configurations that are resiliently flexible, malleable, and/or articulatable (e.g., with a plurality of interconnected rigid links), for example. Though not shown, a proximal end of outer sheath (102) is coupled with a scope base that may be mounted to and controlled by a motorized drive mechanism (e.g., similar to instrument driver (66)) of a robotic arm.

Surgical scope (100) further includes a deflectable distal sheath (106) that extends distally from a distal end of outer sheath (102), and a scope shaft (108) that is slidably disposed within outer sheath (102) and distal sheath (106). Scope shaft (108) includes a deflectable distal shaft portion (110) (also referred to as a “leader”) having an articulation section (112) and a distal tip section (114). Distal tip section (114) includes an optical module having a distally facing lens (not shown) configured to provide visualization of a target surgical site within body cavity (C). The deflectable construction of deflectable distal shaft portion (110) enables it to conform to the curvatures of curved joint (104) and distal sheath (106) as scope shaft (108) slidably advances and retracts relative to outer sheath (102) and distal sheath (106). In some versions, scope shaft (108) may further include a rigid proximal shaft portion that is directly connected to deflectable distal shaft portion (110) and that facilitates insertion and retraction of scope shaft (108). Additionally, in some other versions distal sheath (106) may be an independent structure and used with surgical scope (100).

In the present version, distal sheath (106) and scope shaft (108) cooperate to provide surgical scope (100) with six degrees of freedom, as illustrated by respective arrows in FIG. 7. Specifically, first and second degrees of freedom are provided by the ability of distal sheath (106) to articulate in first and second planes that perpendicularly intersect one another, thus providing pitch and yaw at distal sheath (106). By way of example only, one or more pull-wires, drive bands, and/or other actuation members may be used to drive articulation of distal sheath (106). A third degree of freedom is provided by the ability of scope shaft (108) to longitudinally advance and retract relative to distal sheath (106) and outer sheath (102). Fourth and fifth degrees of freedom are provided by the ability of the deflectable distal shaft portion (110) to articulate at articulation section (112) in first and second planes that perpendicularly intersect one another, thus providing pitch and yaw at articulation section (112). By way of example only, one or more pull-wires, drive bands, and/or other actuation members may be used to drive articulation of distal shaft portion (110). A sixth degree of freedom is provided by the ability of distal tip section (114) to rotate about its longitudinal axis relative to a proximal remainder of deflectable distal shaft portion (110), thus providing roll at distal tip section (114). In some other versions, the components of surgical scope (100) may be modified to include more or fewer (e.g., zero) articulation sections, each of which may be configured to articulate in one or more planes, to provide any desired quantity and arrangement of degrees of freedom. By way of example only, in other versions scope shaft (108) may include zero or two or more articulation sections (112), each configured to articulate in one or more intersecting planes.

In use, a surgeon may first create an incision in body wall (W), for example at the umbilicus, to provide access to a target surgical site located within body cavity (C). One or more other surgical instruments (120) may be inserted through body wall (W) at separate locations, for example each with a surgical cannula or other surgical access device. Each surgical instrument (120) may be mounted to a respective robotic arm and includes an end effector (122) (shown schematically) that is operable to grasp tissue, cut tissue, staple tissue, seal tissue, and/or provide other functionality at the target surgical site. Distal sheath (106) of surgical scope (100) is inserted distally through the incision in body wall (W) into body cavity (C) while outer sheath (102) remains supported by a respective robotic arm. Surgical scope (100) may then be actuated, for example by a drive mechanism of the respective robotic arm, to advance scope shaft (108) distally through outer sheath (102) and distal sheath (106) and into body cavity (C), such that distal sheath (106) serves as an introducer cannula. Before, during, or after advancement of scope shaft (108), distal sheath (106) may be articulated by the drive mechanism to a desired articulated state. Additionally, upon exiting distal sheath (106) and entering body cavity (C), articulation section (112) of scope shaft (108) may be driven by the drive mechanism to orient distal tip section (114) in a desired direction. Additionally, distal tip section (114) may be rotated relative to the proximal remainder of scope shaft (108) to provide desired visualization within body cavity (C).

### III. Surgical Cannula Having Distal Articulation Joint

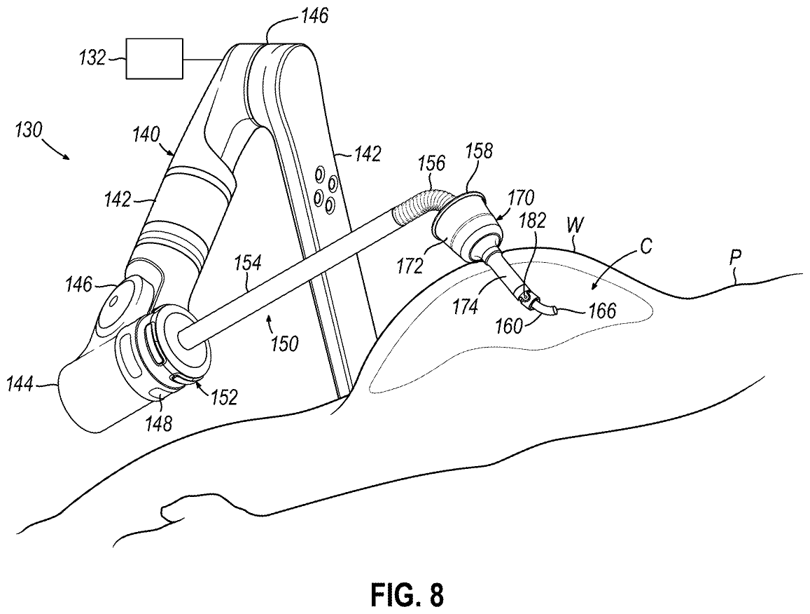

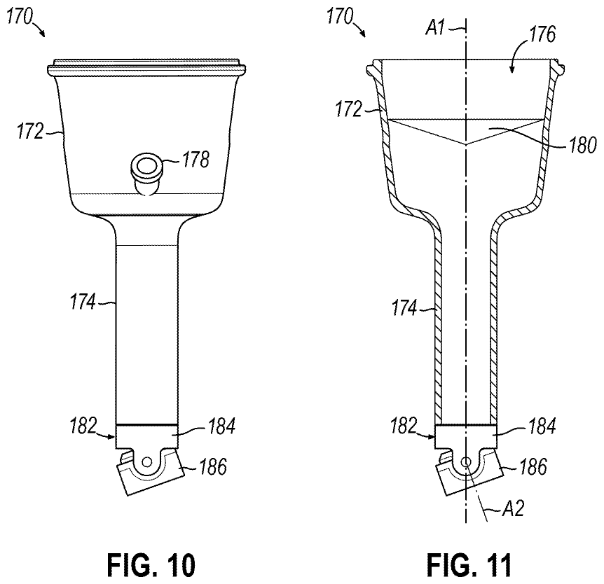

As described above, distal sheath (106) of surgical scope (100) may be configured to articulate in one or more planes to facilitate positioning of distal tip section (114) within body cavity (C) for optimal visualization of a target surgical site and surrounding anatomical structures. In some instances, it may be desirable to combine a deflectable surgical scope with a surgical cannula having a distal articulation feature in the form of an articulation joint. As described below, FIGS. 8-21 show some versions of such surgical cannulas.

FIG. 8 shows an example of a robotic system (130) that includes a robotic arm (140), a surgical instrument in the form of a surgical scope (150) removably coupled to a head (144) of robotic arm (140), and a surgical access device in the form of a cannula (170) coupled with surgical scope (150) remotely from head (144). Robotic arm (140) includes arm segments (142) and head (144) that are interconnected by movable joints (146), where head (144) includes a motorized drive mechanism (148) that may be similar to instrument driver (66) described above. Robotic arm (140) is operable to selectively position and orient surgical scope (150) relative to a patient (P) by driving arm segments (142) and drive mechanism (148) based on control signals received from a master controller (132) of robotic system (130), shown schematically. Master controller (132) may be operatively coupled with robotic arm (140), including surgical scope (150), via a wired connection or a wireless connection, for example.

Robotic arm (140) may be similar in structure and function to any of robotic arms (20, 32) described above, and is mountable to any suitable arm support structure such as a column (22) or any of the other arm support structures disclosed above or in the patent references incorporated by reference herein. Though not shown, robotic system (130) may further include one or more additional robotic arms (140) each supporting and controlling a respective surgical instrument having an end effector of which surgical scope (150) may provide visualization within a body cavity (C) of the patient (P).

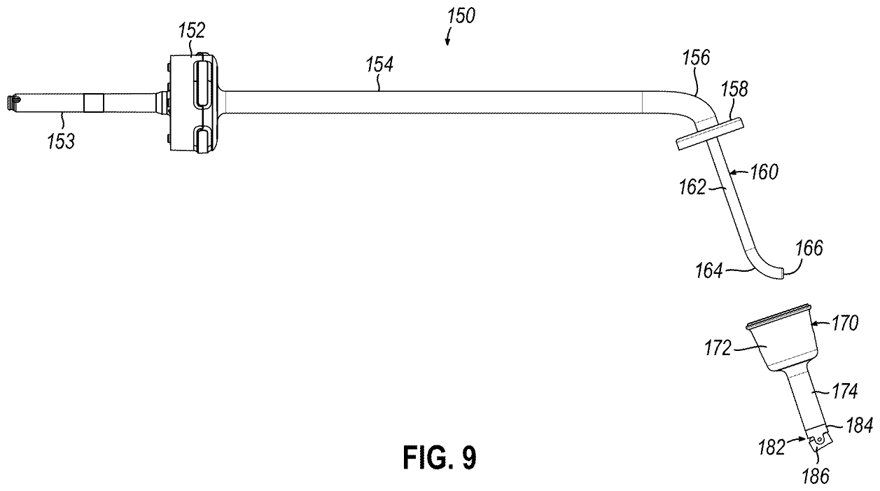

As shown in FIGS. 8 and 9, surgical scope (150) includes a scope base (152), an elongate rigid outer sheath (154) that extends distally from scope base (152), a flexible joint (156) at a distal end of outer sheath (154), a cannula docking plate (158) at a distal end of flexible joint (156), and a scope shaft (160) slidably disposed within outer sheath (154). Flexible joint (156) may have a predefined curved shape or be configured to assume a curved shape as shown and may include an internal sheath (not shown) that resists binding. Similar to scope shaft (108) described above, scope shaft (160) includes a deflectable distal shaft portion (162) that includes an articulation section (164). Scope base (152) may be similar to instrument base (76) described above in that scope base (152) is configured to attach to drive mechanism (148) of robotic arm head (144) such that drive inputs of scope base (152) operatively couple with respective drive outputs of drive mechanism (148). Scope base (152) includes an elongate cylindrical extension (153) (also referred to as a nosecone) that extends coaxially through a central clearance bore (not shown) of drive mechanism (148). Drive mechanism (148) is operable to drive insertion (i.e., longitudinal advancement and retraction) and articulation of scope shaft (160) relative to outer sheath (154). In some versions, drive mechanism (148) may also be further operable to drive articulation of cannula (170), described below.

As shown in FIGS. 8-11, cannula (170) includes a proximal structure in the form of a cup (172) having an open proximal end, and a distal structure in the form of an elongate tube (174) that extends distally from a distal end of cup (172) and has a smaller maximum outer diameter than cup (172). A distal end of cup (172) tapers radially inwardly to the proximal end of tube (174). In the present version, both of cup (172) and tube (174) are rigid such that neither is configured to deflect laterally during use when tube (174) is deployed in a body wall (W). In other versions, all or a portion of tube (174) may be laterally deflectable (e.g., flexible).

As shown in FIG. 11, the interiors of cup (172) and tube (174) cooperate to define a working channel (176) that extends along a central primary axis (A1) of cannula (170) and is sized and configured to receive and guide scope shaft (160) of surgical scope (150) longitudinally therethrough into body cavity (C). A proximal lip of cup (172) is configured to releasably couple with cannula docking plate (158) of surgical scope (150) such that the open proximal end of cup (172) is enclosed by docking plate (158). A distal portion of an annular sidewall of cup (172) includes an access port (178) that communicates with working channel (176). In some versions, access port (178) may be configured to couple with a source of insufflation gas, such as pressurized air, for directing the gas into or out of body cavity (C) to regulate insufflation of body cavity (C). Additionally, or alternatively, access port (178) may be configured to receive various other fluids and/or surgical instruments. Cup (172) further includes an inner seal member (180) (shown schematically) configured to establish an air-tight seal against scope shaft (160) when scope shaft (160) is positioned within working channel (176), and also when scope shaft (160) is removed from working channel (176), to thereby maintain insufflation of body cavity (C) during a procedure. In other versions, cannula (170) and/or surgical scope (150) may include one or more additional seal members configured to maintain insufflation of body cavity (C).

In addition to the foregoing, cannula (170) may be constructed and operable in accordance with at least some of the teachings of U.S. Pat. No. 10,792,069, entitled “Trocar Seal Assemblies,” issued Oct. 6, 2020; U.S. Pat. No. 10,820,924, entitled “Asymmetric Shaft Seal,” issued Nov. 3, 2020; U.S. Pub. No. 2021/0338272, entitled “Pinch to Release Cannula Depth Limiter,” published Nov. 4, 2021; and/or U.S. Pat. No. 10,939,937, entitled “Trocar with Oblique Needle Insertion Portion and Perpendicular Seal Latch,” issued Mar. 9, 2021. The disclosures of these references are incorporated by reference herein, in their entirety.

Cannula (170) further includes an articulation feature in the form of an articulation joint (182) at the distal end of tube (174). Similar to distal sheath (106) of surgical scope (100), articulation joint (182) is configured to facilitate in positioning a distal tip section (166) of surgical scope shaft (160) at a desired location and orientation within body cavity (C) to visualize the target surgical site. Articulation joint (182) of the present example includes a rigid proximal link (184) that is affixed to a distal end of tube (174), and a rigid distal link (186) pivotably coupled with proximal link (184) about a pivot axis that extends transversely to primary axis (A1) of cannula (170). Accordingly, articulation joint (182) of the present version is configured to articulate in a single plane that includes primary axis (A1) to orient distal link (186) along an articulated secondary axis (A2) that is angled relative to primary axis (A1), as shown in FIG. 11. In other versions, distal link (186) may be configured to pivot relative to proximal link (184) about one or more additional pivot axes, and/or articulation joint (182) may include one or more additional links pivotably coupled with proximal and distal links (184, 186) about non-parallel pivot axes, such that articulation joint (182) is configured to articulate in two or more planes that intersect one another and include or extend parallel to primary axis (A1).

Articulation of articulation joint (182) may be active or passive. For instance, articulation joint (182) may be driven by drive mechanism (148) of robotic arm head (144) via one or more articulation drivers (not shown), such as one or more tendons (e.g., pull-wires, drive bands, etc.); or by another drive mechanism positioned remotely from robotic arm head (144), for example as described below. Alternatively, articulation joint (182) may be configured to passively assume an articulated state in response to scope shaft (160) being driven into an articulated state at its articulation section (164). In some such versions, articulation joint (182) may be resiliently biased toward a straight configuration in which distal link (186) is coaxial with primary axis (A1) of cannula (170). In other versions, articulation joint (182) may be manipulated by hand into an articulated state before insertion through body wall (W), and it may be configured to maintain such a preset articulated state. As another variation, another instrument (e.g., grasper) within the cavity (C) may be used to manipulate articulation joint (182) in the patient (P).

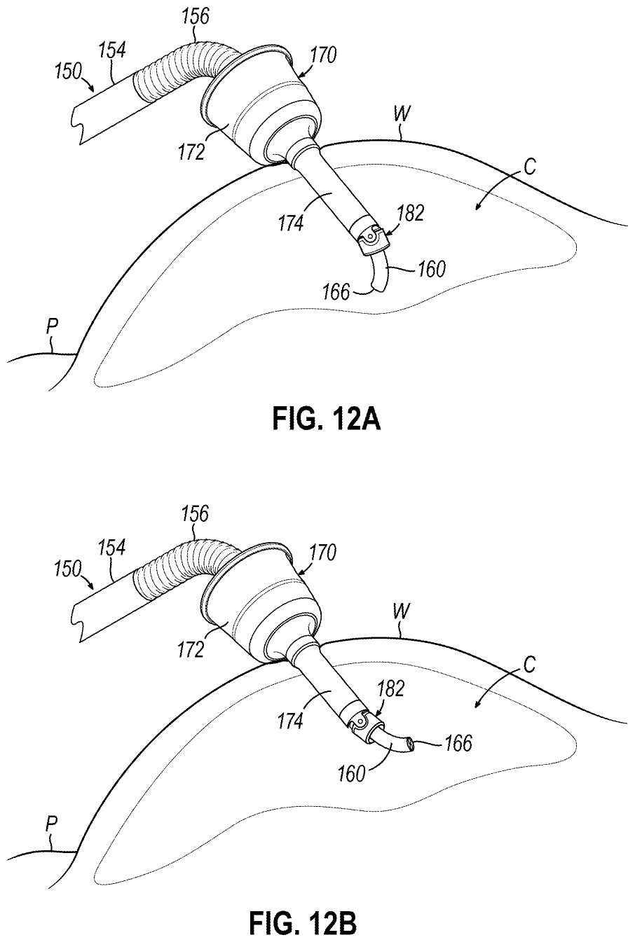

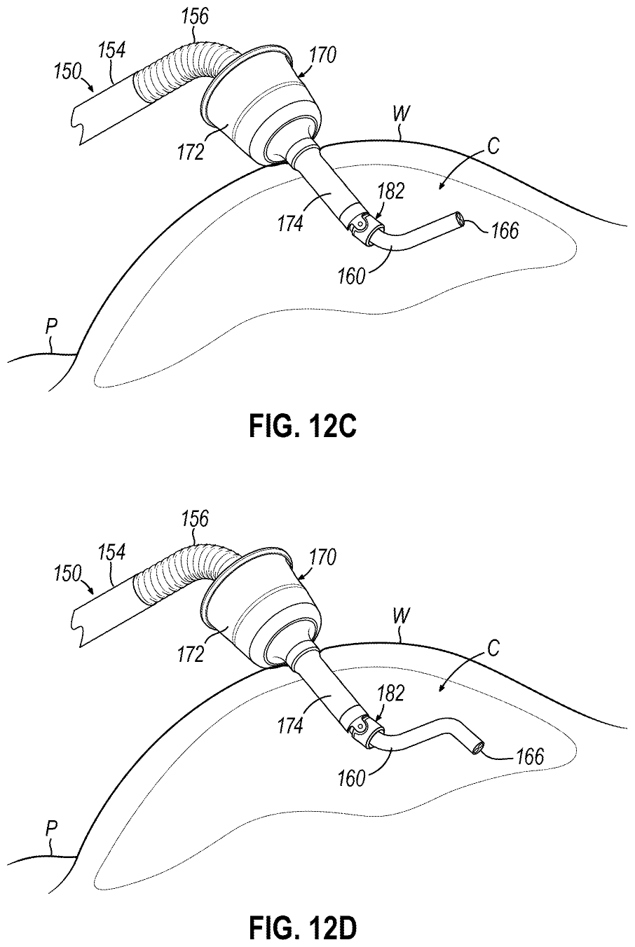

As shown in FIG. 12A, cannula tube (174) is inserted through body wall (W), for example using an obturator (not shown), such that at least the distal end of cannula tube (174) is positioned within body cavity (or “intracorporeally”) and cup (172) is positioned external to body cavity (C) (or “extracorporeally”). In versions where an obturator is used during insertion through body wall (W), the obturator may be removed from cannula tube (174) before surgical scope shaft (160) is inserted through cannula tube (174). As shown in FIG. 8, robotic arm (140) and its head (144) are positioned remotely from cannula (170) such that outer sheath (154) drapes away from cannula (170) via the flexibility of flexible joint (156), thus generally clearing the workspace directly above body wall (W) for use by other robotic arms (not shown) supporting other surgical instruments. In some procedures, cannula (170) may be stabilized relative to the patient with a mechanical grounding feature (not shown), such as a stationary arm. Such a stationary arm may be secured to table (16, 34) or some other grounding structure.

As shown in FIGS. 12A-12D, scope shaft (160) is actuated by drive mechanism (148) distally through cannula (170) and into body cavity (C). FIG. 12A shows articulation joint (182) in a first example of an articulated state in which articulation joint (182) directs scope shaft (160) in a first angled direction within body cavity (C). FIG. 12B shows articulation joint (182) in a second example of an articulated state in which articulation joint (182) directs deflectable scope shaft (160) in a second angled direction within body cavity. As noted above, articulation joint (182) may be configured to transition between such articulated states actively or passively. In cases of active cannula articulation, deflectable distal shaft portion (162) may be flexible along its length to conform to the curvature defined by articulation joint (182) as scope shaft (160) is inserted further into body cavity (C), as shown particularly in FIG. 12C. As shown in FIG. 12D, scope shaft (160) may be articulated at its distal articulation section (164) to suitably orient its distal tip section (166) within body cavity (C) to provide visualization of the target surgical site and/or surrounding anatomical structures. In the example shown in FIG. 12D, scope shaft (160) has achieved a dogleg bend configuration, though scope shaft (160) may alternatively achieve any other suitable kind of articulated state, including but not limited to different articulated states with two or more bends regions at different corresponding positions along the length of scope shaft (160).

### IV. Surgical Scope Systems Having Scope Feed Device

In some instances, during a surgical procedure in which the distal tip of a surgical scope is positioned within the body cavity (C) of a patient, it may be desirable to maximize a field of view of the surgical scope by retracting the distal tip proximally toward the patient's body wall (W). In order for the distal tip to be positioned as such, a distal end of the cannula through which the surgical scope slidably extends may also be retracted proximally toward the body wall (W). Accordingly, it may be desirable to provide a device that is operable to selectively advance and retract the cannula and surgical scope relative to the body wall, through the surgical opening.

### A. Scope Feed Device with Integrated Motor

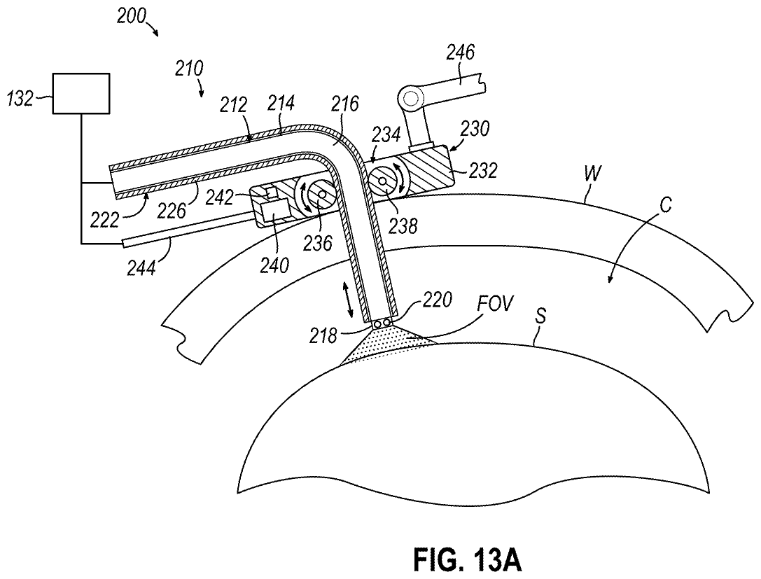

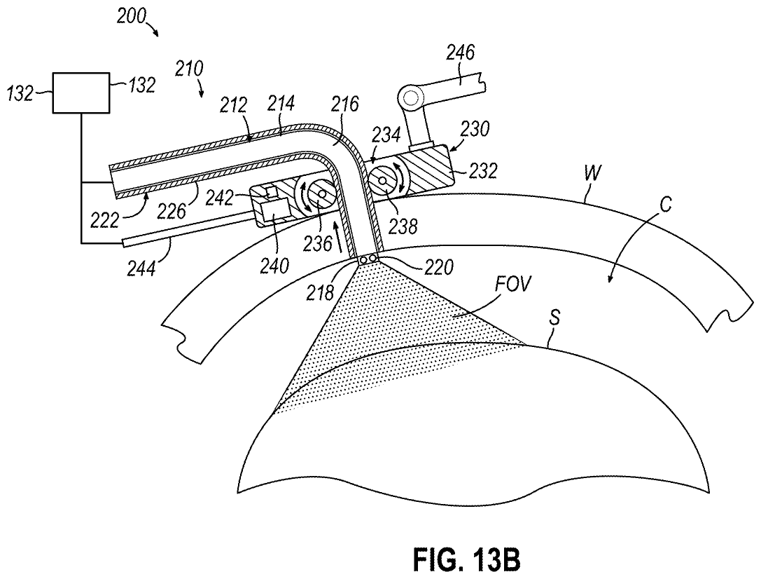

FIGS. 13A-13B show an illustrative surgical scope system (200) that includes a surgical scope assembly (210) and a scope feed device (230) operable to selectively advance and retract surgical scope assembly (210) relative to the body wall (W) of a patient through a surgical opening in the body wall (W). As shown schematically, each of surgical scope assembly (210) and scope feed device (230) is in communication with master controller (132) of robotic system (130).

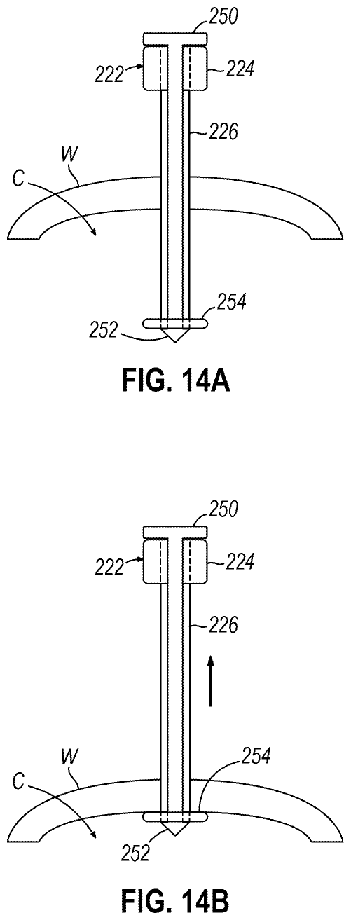

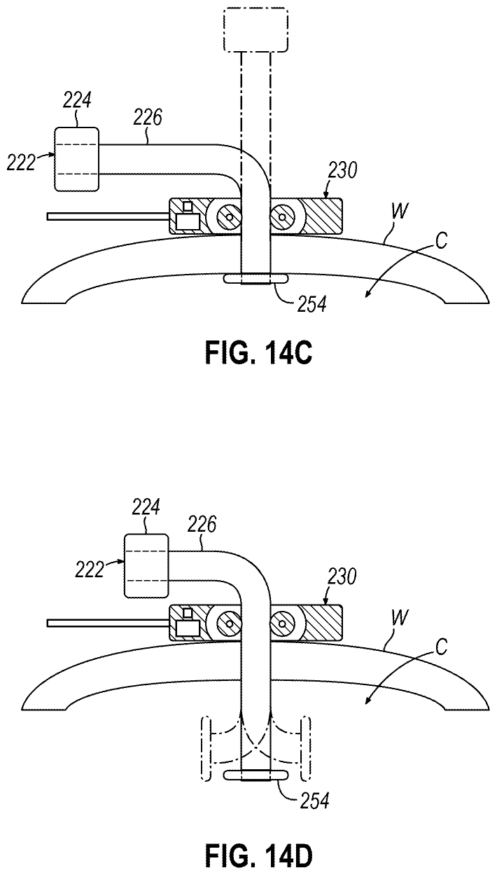

Surgical scope assembly (210) includes a surgical scope (212) having a scope base (not shown) similar to scope base (152) described above and configured to attach to drive mechanism (148) of robotic arm head (144), and a scope shaft (214) that is actuatable by the scope base. Scope shaft (214) includes a deflectable distal shaft portion (216) that may include an articulation section and a distal tip (218), where distal tip (218) includes an optical module having a distally facing lens (220) configured to provide visualization of an anatomical structure (S) within body cavity (C). Surgical scope assembly (210) further includes a cannula (222) having a proximal head (224) (see FIG. 14A) and a deflectable sheath (226) extending distally from head (224). Cannula head (224) may be configured to releasably couple with a proximal sheath structure (not shown) of surgical scope (212), for example as disclosed in U.S. patent application Ser. No. 17/941,059, entitled “Flexible Articulating Introducer Cannula for Surgical Scope in Robotic System,” filed Sep. 9, 2022, the disclosure of which is incorporated by reference herein in its entirety. Cannula sheath (226) is configured to extend distally through a surgical opening in body wall (W) and is further configured to slidably guide scope shaft (214) into and out of body cavity (C). A distal end of cannula sheath (226) may include a driven articulation section configured to articulate within body cavity (C) to selectively orient distal tip (218) of scope shaft (214) relative to anatomical structure (S), for example similar to articulation joint (182) described above.

Scope feed device (230) of the present example includes a device body (232) configured to be positioned directly on an exterior side of body wall (W). Device body (232) includes a feed passage (234) that opens through an upper side and an opposed lower side of device body (232) and is configured to be aligned coaxially with a surgical opening in body wall (W). Feed passage (234) is sized and configured to receive surgical scope assembly (210) therethrough for actuating surgical scope assembly (210) through the surgical opening and relative to body wall (W), as described in greater detail below. Scope feed device (230) further includes a pair of rotary members (236, 238) each rotatable relative to device body (232) about a respective rotational axis. Rotary members (236, 238) extend into feed passage (234) and are configured to directly contact and frictionally engage diametrically opposed sides of surgical scope assembly (210). Rotary members (236, 238) are configured to rotate in opposing rotational directions from one another as surgical scope assembly (210) advances or retracts relative to device body (232). Various alternative quantities and arrangements of rotary members (236, 238) may be included in other versions of scope feed device (230).

An actuator in the form of a motor (240), shown schematically, is housed within device body (232) and is configured to rotatably drive at least one rotary member (236, 238) about its respective rotational axis such that the at least one rotary member (236, 238) functions as a drive member operable to actuate surgical scope assembly (210) relative to device body (232) and body wall (W). In some versions, both rotary members (236, 238) may be driven by motor (240), whereby rotary members (236, 238) rotate in opposing directions as noted above. In all such configurations, rotary members (236, 238) cooperate to advance, retract, and guide surgical scope assembly (210) relative to device body (232) and body wall (W).

Scope feed device (230) further includes a sensor (242), shown schematically, supported by device body (232). In some versions, sensor (242) may be in the form of a rotary encoder that is integrated with or otherwise operatively coupled with motor (240) or a rotary member (236, 238). In such versions, sensor (242) is configured to detect at least one of a rotational position, a direction of rotation, or a speed of rotation of at least one of motor (240) or a rotary member (236, 238) during operation of scope feed device (230). Sensor (242) may then provide an electrical signal to master controller (132) so that master controller (132) may track continuous variable positioning of surgical scope assembly (210) relative to device body (232). In other versions, sensor (242) may be configured to detect two or more predetermined discrete positions of surgical scope assembly (210) relative to device body (232), such as a fully advanced position (e.g., see FIG. 13A) and a fully retracted position (e.g., see FIG. 13B), and optionally one or more intermediate discrete positions therebetween. In such versions, sensor (242) my include or otherwise communicate with one or more mechanical switches, hall effect sensors, or reed switches, for example. As shown, scope feed device (230) includes a connection cable (244) that electrically couples motor (240), sensor (242), and any other electrical components housed by device body (232) with master controller (132), as well as a motor power source (not shown) if located remotely from device body (232).

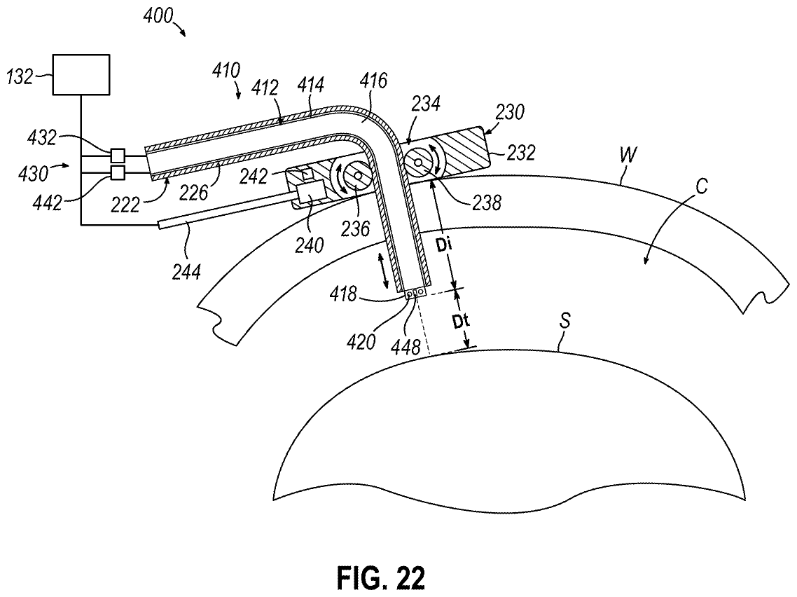

Based on a signal provided by sensor (242), master controller (132) may be configured to determine an amount by which surgical scope assembly (210) has been advanced by scope feed device (230) relative to device body (232) and body wall (W). In other words, master controller (132) is configured to determine a magnitude and direction of longitudinal actuation of surgical scope assembly (210) by scope feed device (230). As described in greater detail below in connection with FIGS. 22-24, this information may be used by master controller (132) to determine an insertion depth of distal tip (218) of surgical scope (212) within body cavity (C), which may then be used by master controller (132) to control scope feed device (230) to locate distal tip (218) at a threshold distance from anatomical structure (S).

It will be appreciated that the engagement between scope feed device (230) and surgical scope assembly (210) may operate to stabilize surgical scope assembly (210) relative to body wall (W) at the surgical opening. Optionally, scope feed device (230) itself may be stabilized relative to body wall (W) by a static arm (246), which may be connected to a structure remote from device body (232), such as a side rail of surgical table (16, 34), for example.

FIG. 13A shows surgical scope assembly (210) in an illustrative distally advanced position within body cavity (C) in which distal tip (218) of surgical scope (212) is spaced distally from an interior side of body wall (W). In this position, distal tip (218) has a field of view (FOV) that may be too small for the surgeon to adequately view anatomical structure (S) and surrounding regions within body cavity (C). FIG. 13B shows surgical scope assembly (210) after it has been retracted proximally by scope feed device (230) such that the distal end of cannula sheath (226) is fully within body wall (W) and distal tip (218) of surgical scope (212) is just distal to the interior side of body wall (W). This positioning provides distal tip (218) with a substantially larger field of view (FOV) that provides the surgeon with enhanced visualization within body cavity (C) such that the surgeon may view more of anatomical structure (S) and adjacent regions without having to articulate surgical scope (212) within body cavity (C).

As described above, scope feed device (230) is operable to selectively advance and retract surgical scope assembly (210), which includes both surgical scope (212) and cannula (222). More specifically, scope feed device (230) is operable to longitudinally actuate scope shaft (214) and cannula sheath (226) in combination relative to body wall (W). It will be appreciated that scope shaft (214) is slidably disposed within cannula sheath (226) such that scope shaft (214) may still be actuated longitudinally relative to cannula (222) by a separate drive mechanism, such as motorized drive mechanism (148) of robotic arm (140), for example.

FIGS. 14A-14D show an illustrative process for setting up surgical scope system (200) and setting an initial depth of cannula (222) relative to the body wall (W) of a patient. As shown in FIG. 14A, an obturator (250) having a pointed distal end (252) is inserted into the lumen of cannula (222) such that pointed distal end (252) is exposed at a distal end of cannula sheath (226). The distal end of cannula sheath (226) includes an annular retention feature (254) that protrudes radially. The combined cannula (222) and obturator (250) are directed distally through body wall (W) so that their distal ends enter body cavity (C) through a surgical opening. The surgical opening may be formed with a knife prior to insertion of cannula (222) and obturator (250), or it may be formed by the pointed distal end (252) of obturator (250) during insertion, for example with a rotational motion. Retention feature (254) of cannula (222) may flex as it passes through body wall (W) and then resiliently return to its original form upon entering body cavity (C). In other versions, retention feature (254) may be movable between a retracted state and a deployed state, and it may assume the deployed state upon or shortly after entering body cavity (C). As shown in FIG. 14B, the combined cannula (222) and obturator (250) are pulled proximally by the surgeon until retention feature (254) abuts the interior side of body wall (C), which provides a tactile indication to the surgeon that the proper initial insertion depth of cannula (222) has been reached. Upon detecting this position, the surgeon then removes obturator (250) from cannula (222). In other versions, retention feature (254) may be omitted from cannula (222)

Scope feed device (230) may be positioned on body wall (W) before or after insertion of cannula (222). For instance, scope feed device (230) may first be positioned on body wall (W) such that feed passage (234) aligns coaxially with the intended point of insertion through body wall (W). Then, the combined cannula (222) and obturator (250) may be directed distally through feed passage (234) and through body wall (W) in the manner described above. Alternatively, cannula (222) may first be positioned in the manner described above, and then scope feed device (230) may be positioned around the external portion of cannula sheath (226), for example by directing scope feed device (230) over the proximal end of cannula (222), with or without first detaching cannula head (224) from cannula sheath (226). In either scenario, scope feed device (230) is ultimately arranged coaxially with cannula (222) as shown in FIG. 14C. Additionally, upon the removal of obturator (250) from cannula (222), the proximal end of cannula (222) may be draped laterally away from scope feed device (230), as shown in FIG. 14C. As shown in FIG. 14D, cannula (222) may then be actuated longitudinally relative to body wall (W) by scope feed device (230). Additionally, the distal end of cannula sheath (226) may be articulated at its articulation section within body cavity (C). Such articulation may be driven by a separate drive mechanism, such as motorized drive mechanism (148) of robotic arm (140) or by a user-actuated feature (not shown), for example as disclosed in U.S. patent application Ser. No. 17/941,063, entitled “Articulating Introducer Cannula for Endoscope in Robotic System,” filed Sep. 9, 2022, the disclosure of which is incorporated by reference herein, in its entirety.

### B. Scope Feed Devices with Remote Motor

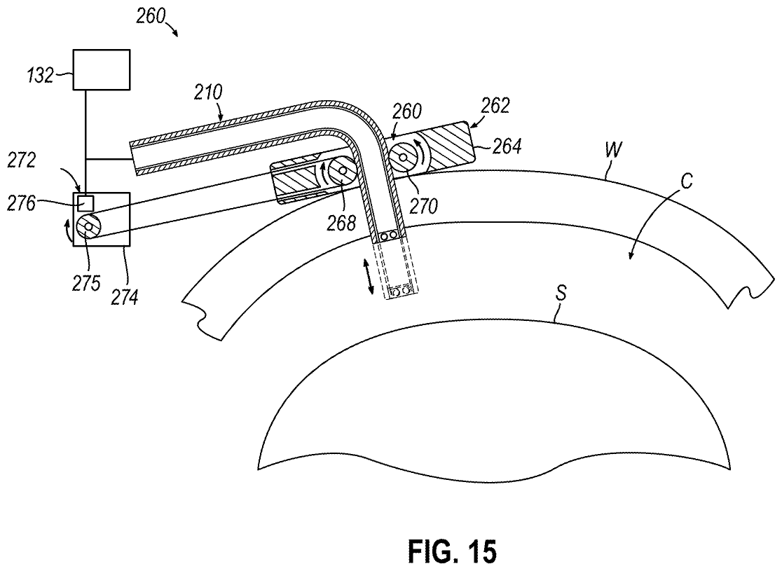

In some instances, it may be desirable to locate the motor of scope feed device (230) remotely from device body (232). FIGS. 15-17 show several illustrative systems that incorporate such a variation of scope feed device (230). Each such system is similar to surgical scope system (200) described above except as otherwise described below.

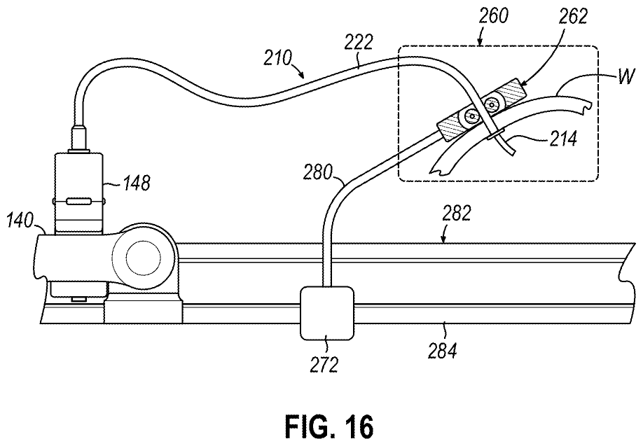

FIGS. 15 and 16 show an illustrative surgical scope system (260) that includes surgical scope assembly (210) and a scope feed device (262) operable to selectively advance and retract surgical scope assembly (210) relative to the body wall (W) of a patient. Similar to scope feed device (230) described above, scope feed device (262) includes a device body (264) having a feed passage (266) configured receive surgical scope assembly (210) therethrough. Device body (264) houses a pair of rotary members (268, 270) configured to engage and drive surgical scope assembly (210) proximally and distally through feed passage (266) relative to body wall (W).

Unlike scope feed device (230), scope feed device (262) includes a motor unit (272) located remotely from device body (264). For example, as shown in FIG. 16, motor unit (272) may be secured to a side rail (284) of a surgical bed (282). As shown schematically in FIG. 15, motor unit (272) includes a motor (274), a motor pulley (275), and a sensor (276). Motor pulley (275) is operatively coupled with at least one rotary member (268, 270) within device body (264) by an elongate power transmission member in the form of a drive cable (286), such that rotation of motor (274) drives rotation of motor pulley (275) and the at least one rotary member (268, 270), which in turn actuates surgical scope assembly (210) through feed passage (266). As shown in FIG. 16, drive cable (286) may be slidably housed within an elongate outer sheath (280) that extends between device body (264) and motor unit (272), and which may be rigid or flexible. Sensor (276) may be similar to sensor (242) described above and may be in the form of or otherwise include or communicate with a rotary encoder or one or more mechanical switches, hall effect sensors, or reed switches, for example. Sensor (276) is in communication with master controller (132) and is configured to enable master controller (132) to determine a magnitude and direction of longitudinal actuation of surgical scope assembly (210) by scope feed device (262).

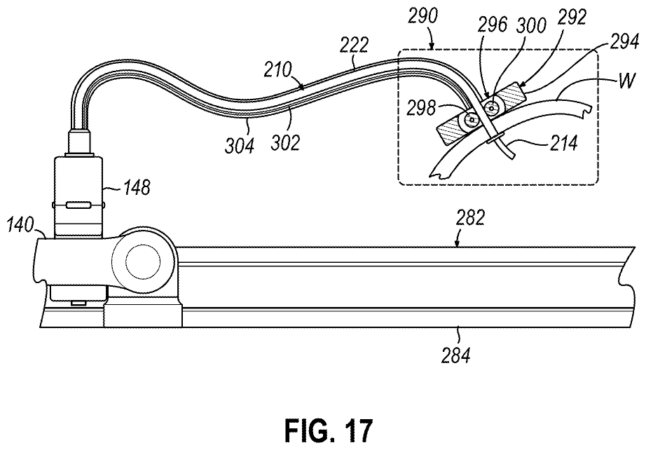

FIG. 17 shows another illustrative surgical scope system (290) that includes surgical scope assembly (210) and a scope feed device (292) operable to selectively advance and retract surgical scope assembly (210) relative to the body wall (W) of a patient. Scope feed device (292) includes a device body (294), a feed passage (296), and a pair of rotary members (298, 300). In this example, rotary members (298, 300) of scope feed device (292) are driven by motorized drive mechanism (148) of robotic arm (140) via a drive cable (302), where drive mechanism (148) additionally drives longitudinal actuation of scope shaft (214) through cannula sheath (226). An outer sheath (304) extends between scope feed device (292) and drive mechanism (148) such that outer sheath slidably houses each of scope shaft (214) and drive cable (302). Though not shown, a sensor similar to sensors (242, 276) may be integrated into one or both of device body (294) and drive mechanism (148) and may be configured to communicate with master controller (132) to enable master controller (132) to determine a magnitude and direction of longitudinal actuation of surgical scope assembly (210) by scope feed device (292).

### C. Scope Feed Devices with User-Actuated Drive Member

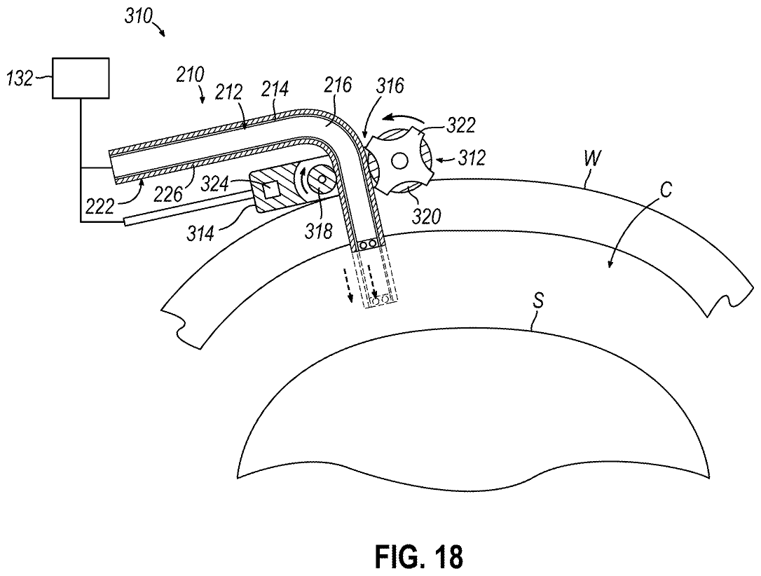

As discussed above in connection with scope feed devices (230, 262, 292), surgical scope assembly (210) is advanced and retracted relative to body wall (W) by an actuator in the form of a motor. In some instances, it may be desirable to provide a scope feed device with an actuator in the form of a user-actuatable feature that allows for manual advancement and retraction of surgical scope assembly (210). FIGS. 18-21B show illustrative versions of such configurations, as described in greater detail below.

FIG. 18 shows an illustrative surgical scope system (310) that includes surgical scope assembly (210) and a scope feed device (312) operable to selectively advance and retract surgical scope assembly (210) relative to the body wall (W) of a patient. Similar to scope feed devices (230, 262, 292) described above, scope feed device (312) includes a device body (314) having a feed passage (316) configured receive surgical scope assembly (210) therethrough. Device body (314) supports a pair of rotary members (318, 320) configured to engage and drive surgical scope assembly (210) proximally and distally through feed passage (266) relative to body wall (W). Rotary members (318, 320) are rotatable in opposite directions about respective rotational axes.

Unlike second rotary members (238, 270, 300) described above, second rotary member (320) is larger in diameter than first rotary member (318) and is operatively coupled with an actuator in the form of a knob (322) configured to be gripped and rotated by a surgeon to manually rotate second rotary member (320) relative to device body (314). Knob (322) may be integral with or rigidly attached to second rotary member (320) as shown in FIG. 18. In other versions, knob (322) may be separate from but operatively coupled with second rotary member (320), for example with one or more power transmissions members such as gears, cables, belts, and the like such that rotation of knob (322) drives rotation of second rotary member (320). Such rotation of second rotary member (320) thus actuates surgical scope assembly (210) longitudinally relative to device body (314), through feed passage (316). Manually operable actuators of various other types may be incorporated in place of knob (322) in other versions. Scope feed device (312) further includes a sensor (324), which may be similar to sensors (242, 276) described above and is configured to communicate with master controller (132) to enable master controller (132) to determine a magnitude and direction of longitudinal actuation of surgical scope assembly (210) by scope feed device (312).

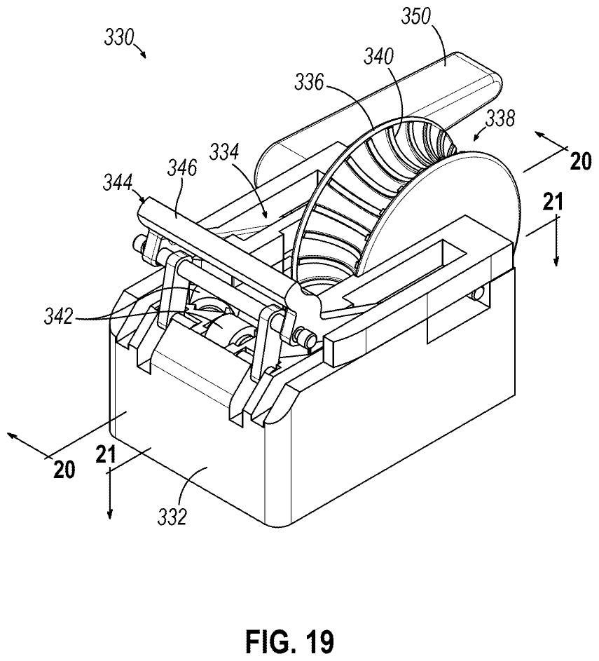

FIGS. 19-21B show another illustrative scope feed device (330) operable to selectively advance and retract surgical scope assembly (210) relative to the body wall (W) of a patient via manual actuation by a surgeon. Scope feed device (330) includes a device body (332) having a feed passage (334) configured to receive surgical scope assembly (210) therethrough. A primary rotary member (336) in the form of a drive wheel is rotatably coupled to device body (332) and includes a concave annular channel (338) about its outer circumference that is sized and shaped to receive surgical scope assembly (210). A plurality of ribs (340) are spaced apart circumferentially within concave annular channel (338) and are configured to facilitate frictional engagement with surgical scope assembly (210). An array of secondary rotary members (342) in the form of rollers of smaller diameter are rotatably coupled with device body (332) on an opposing side of feed passage (334). Each rotary member (336, 342) is rotatable relative to device body (332) about a respective rotational axis.

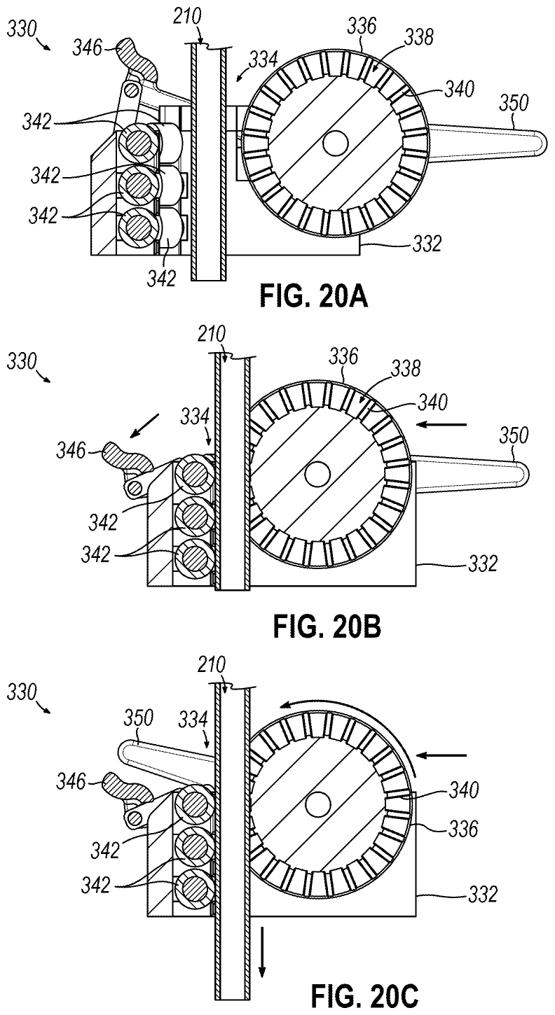

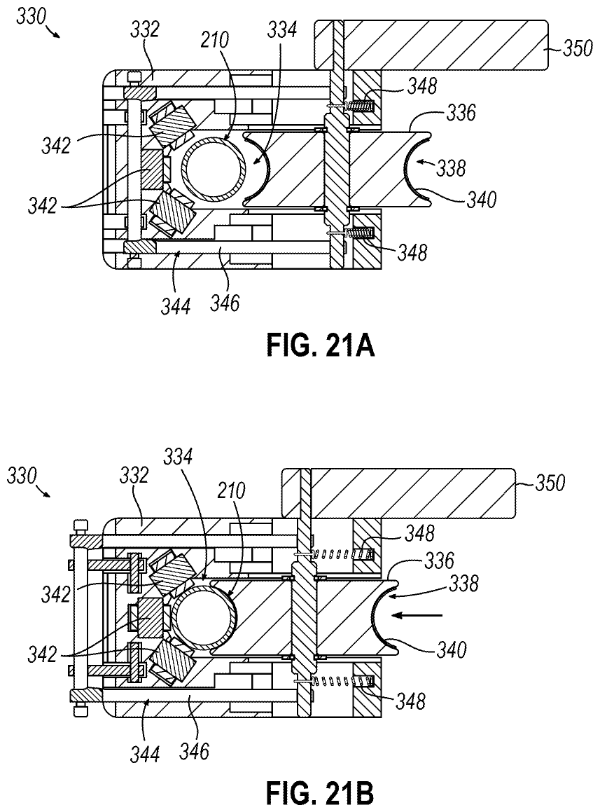

Scope feed device (330) further includes a latch mechanism (344) operable to releasably clamp rotary members (336, 342) against surgical scope assembly (210) within feed passage (334). Latch mechanism (344) includes a latch lever (346) movably coupled with device body (332) and actuatable by a surgeon between an unlatched state shown in FIGS. 19, 20A, and 21A, and a latched state shown in FIGS. 20B and 21B. Latch lever (346) may be resiliently biased toward the unlatched state by a pair of resilient members shown in the form of extension springs (348), shown in FIGS. 21A-21B. Latch lever (346) is coupled with an axle of primary rotary member (336) by extension springs (348) such that actuation of the latch lever (346) from the unlatched state to the latched state advances primary rotary member (336) toward secondary rotary members (342), in a direction transverse to a central axis of feed passage (334), to reduce an effective diameter of feed passage (334) and thereby clamp surgical scope assembly (210) between primary rotary member (336) and secondary rotary members (342). Accordingly, rotation of primary rotary member (336) in the latched state drives longitudinal actuation of surgical scope assembly (210) relative to device body (332), and rotation of secondary rotary members (342) in a direction opposite that of primary rotary member (336). It will be appreciated that extension springs (348) function as a force-limiting feature to limit a compression force exerted by rotary members (336, 342) when latch lever (346) is placed in the latched state.

Scope feed device (330) further includes an actuator in the form of a drive lever (350) rigidly coupled with primary rotary member (336). Drive lever (350) is configured to be gripped by a surgeon and rotated to drive rotation of primary rotary member (336) and thus longitudinal actuation of surgical scope assembly (210) relative to device body (332). In the present version, as shown in FIGS. 20B and 20C, rotation of a free end of drive lever (350) in a direction toward a free end of latch lever (346) in the latched state advances surgical scope assembly (210) distally. Conversely, rotation of the free end of drive lever (350) in a direction away from the free end of latch lever (346) in the latched state retracts surgical scope assembly (210) proximally. In the present version, primary rotary member (336) rotates slightly less than 180 degrees as drive lever (350) is moved between these two states. If additional advancement or retraction of surgical scope assembly (210) is desired, latch mechanism (344) may be opened and drive lever (350) may be reset, and then latch mechanism (344) may be reclosed so that drive lever (350) may be rotated again in the direction desired. While the actuator of scope feed device (330) is shown in the form of lever (350), a manual actuator of various other types may be utilized in other versions of scope feed device (330). Though not shown, scope feed device (330) may further include a sensor similar to sensors (242, 276) configured to communicate with master controller (132) to enable master controller (132) to determine a magnitude and direction of longitudinal actuation of surgical scope assembly (210) by scope feed device (330).

### V. Surgical Scope System Having Scope Feed Device and Optical Distance Measurement System

As described above, each scope feed device (230, 262, 292, 312, 330) may incorporate a sensor that communicates with master controller (132) to enable master controller (132) to determine a magnitude and direction of longitudinal actuation of surgical scope assembly (210) by scope feed device (230, 262, 292, 312, 330). In some instances, it may be desirable to further provide a surgical scope system with the ability to measure in real-time a distance from the distal tip of the surgical scope to a target anatomical structure (S) within the body cavity (C). In such systems, master controller (132) may control the scope feed device to locate the distal tip at a threshold distance from the anatomical structure (S). FIGS. 22-24 show an illustrative surgical scope system (400) that is configured in such a manner, and a related method (480) of operation, as described in greater detail below.

### A. Overview of Surgical Scope System

As shown in FIG. 22, surgical scope system (400) includes scope feed device (230) and a surgical scope assembly (410) that is similar to surgical scope assembly (210) described above except as otherwise described below, where each of scope feed device (230) and surgical scope assembly (410) is in communication with master controller (132). It will be appreciated that scope feed device (230) is shown for illustrative purposes only and that any of scope feed devices (262, 292, 312, 330) or variations thereof may be utilized in other versions of surgical scope system (400). Like surgical scope assembly (210), surgical scope assembly (410) includes cannula (222) and a surgical scope (412). Surgical scope (412) includes a scope shaft (414) with a deflectable distal shaft portion (416) and a distal tip (418) that includes a lens (420) configured to provide visualization within the body cavity (C) of a patient.

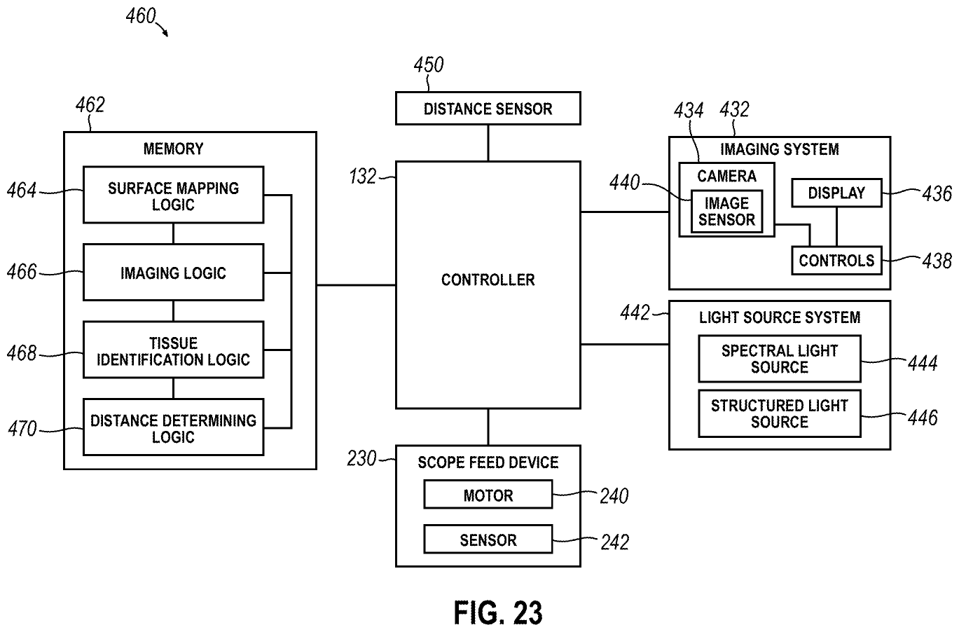

Surgical scope system (400) further includes an optical distance measurement system (430) operable to optically measure a present distance between distal tip (418) and anatomical structure (S), referred to herein as a tissue distance (Dt). Optical distance measurement system (430) of the present example is integrated into surgical scope (412) and includes an imaging system (432) and a light source system (442), shown schematically, components of which may be housed within distal tip (418) in some versions despite the illustration provided in FIG. 22. Optical distance measurement system (430) further includes a distal tip feature (448) positioned within distal tip (418) of surgical scope (412). In some versions, distal tip feature (448) may be in the form of a distance sensor (450) (shown schematically in FIG. 23), such as a time of flight sensor, that communicates with master controller (132). In other versions, distal tip feature (448) may be in the form of a light emitter of light source system (442) that is configured to project light, such as a structured light pattern, onto the anatomical structure (S). In such versions, a camera (434) of imaging system (432) (shown schematically in FIG. 23) is configured to detect the projected light and communicate with master controller (132).

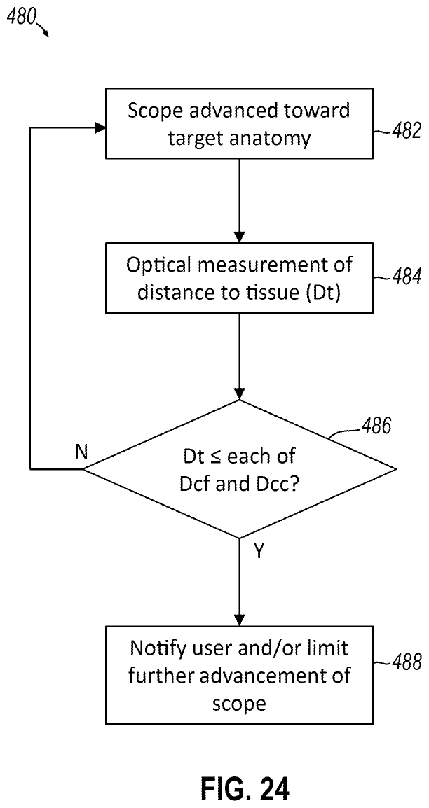

Based on an electrical signal provided by distance sensor (450) or camera (434), master controller (132) is configured to determine a present tissue distance (Dt) between distal tip (418) and anatomical structure (S). Additionally, sensor (242) of scope feed device (230) enables master controller (132) to determine a distance between distal tip (418) and device body (232), referred to herein as an insertion distance (Di), based on the detected magnitude and direction of actuation of surgical scope assembly (410) by scope feed device (230) as tracked by sensor (242). As described in greater detail below in connection with FIG. 24, master controller (132) may reference these distances to locate distal tip (418) at a threshold tissue distance (Dt), or at least track distal tip (418) relative to such a threshold tissue distance (Dt).

### B. Illustrative Control System

FIG. 23 shows a schematic diagram of an illustrative control system (460) that incorporates master controller (132) and surgical scope system (400). Control system (460) may be further configured in accordance with the teachings of U.S. patent application Ser. No. 17/375,281, entitled “Scene Adaptive Endoscopic Hyperspectral Imaging System,” filed Jul. 14, 2021, the disclosure of which is incorporated by reference herein, in its entirety.

In the present version, control system (460) includes master controller (132) in signal communication with a memory (462). Memory (462) stores instructions executable by master controller (132) to determine and/or recognize anatomical structures, such as anatomical structure (S), determine and/or compute one or more distances and/or three-dimensional digital representations, and to communicate certain information to one or more surgeons. For example, memory (462) may store surface mapping logic (464), imaging logic (466), tissue identification logic (468), or distance determining logic (470), or any combinations of logic (464, 466, 468, 470). Control system (460) also includes imaging system (432) having one or more cameras (434), which may be components of surgical scope (412), one or more displays (436), one or more controls (438), or any combinations of these elements. The one or more cameras (434) may include one or more image sensors (440) configured to receive signals from various light sources emitting light at various visible and invisible spectra (e.g., visible light, spectral imagers, three-dimensional lens, among others). Display (436) may include one or more screens or monitors for depicting real, virtual, and/or virtually-augmented images and/or information to one or more clinicians.

In various aspects, a main component of a camera (434) includes an image sensor (440). Image sensor (440) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. Image sensor (440) may also include any suitable number of chips.

Control system (460) of the present example also includes a spectral light source (444) and a structured light source (446). In certain instances, a single light source may be pulsed to emit wavelengths of light in the spectral light range and wavelengths of light in the structured light range. Alternatively, a single light source may be pulsed to provide light in the invisible spectrum (e.g., infrared spectral light) and wavelengths of light on the visible spectrum. A spectral light source (444) may include a hyperspectral light source, a multispectral light source, a fluorescence excitation light source, and/or a selective spectral light source, for example. In various instances, tissue identification logic (468) may identify anatomical structure (S) via data from a spectral light source (444) received by the image sensor (440) portion of camera (434). Surface mapping logic (464) may determine the surface contours of the visible tissue based on reflected structured light. With time-of-flight measurements, distance determining logic (470) may determine one or more distance(s) to the visible tissue and/or anatomical structure (S). One or more outputs from surface mapping logic (464), tissue identification logic (468), and distance determining logic (470), may be provided to imaging logic (466), and combined, blended, and/or overlaid to be conveyed to a surgeon via display (436) of imaging system (432).

### C. Illustrative Method of Operating Surgical Scope System

FIG. 24 shows an illustrative method (480) of operating surgical scope system (400) described above. Surgical scope system (400), including scope feed device (230) and surgical scope assembly (210), may first be set up following the steps disclosed above in connection with FIGS. 14A-14D. At step (482), master controller (132) activates motor (240) of scope feed device (230) to advance surgical scope assembly (410) distally toward anatomical structure (S). At step (484), continuously or periodically throughout the distal advancement of surgical scope assembly (410), optical distance measurement system (430) measures the present tissue distance (Dt) from distal tip (418) of surgical scope (412) to anatomical structure (S) and communicates this information to master controller (132).

At step (486), master controller (132) evaluates whether the present tissue distance (Dt) is less than or equal to a threshold distance. For instance, the threshold distance may be a distance critical for focusing of camera (434) (Dcf) or a minimum distance critical for avoiding contact of distal tip (418) with tissue (Dcc) within body cavity (C). If master controller (132) determines that the present tissue distance (Dt) is not less than or equal to the threshold distance (i.e., the present tissue distance is larger than the threshold distance), then controller (132) returns to step (482) and controls scope feed device (230) to continue advancing surgical scope assembly (410) distally toward anatomical structure (S). Optionally, master controller (132) may also provide a notification to the surgeon that the threshold distance has not yet been reached, for example via display (436). If master controller (132) determines that the present tissue distance (Dt) is less than or equal to the threshold distance, then controller (132) proceeds to step (488). At step (488), master controller (132) may halt longitudinal advancement of surgical scope assembly (410) and/or provide a notification to the surgeon that the threshold distance has been reached or exceeded, for example via display (436). In some versions, if master controller (132) determines that the present tissue distance (Dt) is less than the threshold distance, then controller (132) may control scope feed device (230) to retract surgical scope assembly (410) proximally away from anatomical structure (S).

In any such versions of method (480), master controller (132) may track an insertion distance (Di) of distal tip (418) of surgical scope (412) from device body (232) of scope feed device (230), based on signals provided by sensor (242) of scope feed device (230). Additionally, master controller (132) may determine that the present tissue distance (Dt) differs from the threshold distance by a difference. Master controller (132) may then control scope feed device (230) to distally advance or proximally retract surgical scope assembly (410) by a distance equal to the difference, and simultaneously monitor such advancement or retraction based on signals provided by sensor (242). Controller (132) may then halt further longitudinal actuation of surgical scope assembly (410) upon determining that surgical scope assembly (410) has been advanced or retracted by the requisite distance. In this manner, master controller (132) may position distal tip (418) of surgical scope (412) at the threshold distance from anatomical structure (S). It will be appreciated that at any point before, during, or after performance of the steps discussed above, master controller (132) may be configured to adjust the threshold distance based on a user input, which may be provided via display (436) or controls (438), for example.

### VI. Miscellaneous

The teachings herein may be combined with any one or more of the teachings disclosed in U.S. patent application Ser. No. 17/941,063, entitled “Articulating Introducer Cannula for Surgical Scope in Robotic System,” filed Sep. 9, 2022, the disclosure of which is incorporated by reference herein in its entirety; U.S. patent application Ser. No. 17/941,062, entitled “Bent Introducer Cannula for Surgical Scope in Robotic System,” filed Sep. 9, 2022, the disclosure of which is incorporated by reference herein in its entirety; U.S. patent application Ser. No. 17/941,059 entitled “Flexible Articulating Introducer Cannula for Surgical Scope in Robotic System,” filed Sep. 9, 2022, the disclosure of which is incorporated by reference herein in its entirety; U.S. patent application Ser. No. 17/941,057, entitled “Remotely Driven Camera in Robotic System,” filed Sep. 9, 2022, the disclosure of which is incorporated by reference herein in its entirety; and/or U.S. Pat. App. No. [Atty. Ref. No. AUR6281USNP1], entitled “System and Method to Control Camera Relative to Instruments in Robotic System,” filed on even date herewith, the disclosure of which is incorporated by reference herein in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof are placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1-20. (canceled)

21. A system, comprising:
(a) a surgical scope configured to extend through a surgical opening in a body wall of a patient and into a body cavity, wherein the surgical scope includes a distal tip having a lens configured to visualize an anatomical structure within the body cavity;
(b) a feed device operable to selectively advance and retract the surgical scope relative to the body wall; and
(c) a controller in communication with the surgical scope and the feed device, wherein the controller is configured to:
(i) determine a present target distance measured from the distal tip to the anatomical structure,
(ii) compare the present target distance to a threshold target distance, and
(iii) based on the comparison, at least one of:
(A) control the feed device to advance, retract, or halt the surgical scope longitudinally relative to the body wall, or
(B) provide a notification to a user of the system.

22. The system of claim 21, further comprising a sheath configured to be inserted through the surgical opening and slidably receive the surgical scope, wherein the feed device is operable to selectively advance and retract the surgical scope and the sheath collectively relative to the body wall.

23. The system of claim 22, wherein a distal end of the sheath includes an articulation section configured to articulate within the body cavity to selectively orient the distal tip of the surgical scope relative to the anatomical structure, wherein the surgical scope includes a deflectable distal shaft portion configured to slidably advance and retract through and relative to the sheath.

24. The system of claim 21, further comprising an optical distance measurement system operable to measure the present target distance.

25. The system of claim 24, wherein the optical distance measurement system includes at least one of a sensor or a light emitter arranged at the distal tip of the surgical scope.

26. The system of claim 25, wherein the optical distance measurement system includes a time of flight sensor arranged at the distal tip, wherein the controller is configured to determine the present target distance based on a signal provided by the time of flight sensor.

27. The system of claim 25, wherein the optical distance measurement system includes:
(i) a light emitter arranged at the distal tip and configured to project light onto the anatomical structure, and
(ii) a camera configured to detect the light,
wherein the controller is configured to determine the present target distance based on a signal provided by the camera.

28. The system of claim 27, wherein the light emitter is configured to project a structured light pattern onto the anatomical structure, wherein the camera is configured to detect the structured light pattern.

29. The system of claim 21, wherein the controller is further configured to:
(i) when the present target distance is unequal to the threshold target distance, at least one of:
(A) control the feed device to advance or retract the surgical scope relative to the body wall, or
(B) provide a notification to the user, and
(ii) when the present target distance is equal to the threshold target distance, at least one of:
(A) control the feed device to halt the surgical scope longitudinally relative to the body wall, or
(B) provide a notification to the user.

30. The system of claim 29, wherein when the present target distance is greater than the threshold target distance, the controller is configured to control the feed device to advance the surgical scope distally relative to the body wall.

31. The system of claim 21, wherein the controller is configured to adjust the threshold target distance based on a user input.

32. The system of claim 21, wherein the feed device comprises:
(i) a device body configured to overlie the surgical opening in the body wall, wherein the device body defines a feed passage configured to receive the surgical scope therethrough,
(ii) a drive member that extends into the feed passage, and
(iii) an actuator operable to actuate the drive member to selectively advance and retract the surgical scope relative to the body wall.

33. The system of claim 32, wherein the feed device further includes a sensor configured to detect at least one of a position, a direction of motion, or a speed of motion of at least one of the drive member or the actuator, wherein the controller is configured to determine an actuation magnitude of the surgical scope relative to the device body based on a signal provided by the sensor.

34. The system of claim 33, wherein when the present target distance is greater than the threshold target distance by a difference, the controller is configured to control the feed device to advance the surgical scope distally relative to the body wall by an actuation magnitude equal to the difference.

35. The system of claim 33, wherein the drive member comprises a rotary drive member, wherein the sensor comprises a rotary encoder.

36. A system, comprising:
(a) a surgical scope configured to extend through a surgical opening in a body wall of a patient and into a body cavity, wherein the surgical scope includes a distal tip having a lens configured to visualize an anatomical structure within the body cavity;
(b) an optical distance measurement system operable to measure a present target distance from the distal tip to the anatomical structure;
(c) a feed device operable to selectively advance and retract the surgical scope relative to the body wall; and
(d) a controller in communication with each of the surgical scope, the optical distance measurement system, and the feed device, wherein the controller is configured to:
(i) compare the present target distance to a threshold target distance,
(ii) based on the comparison, at least one of:
(A) control the feed device to advance, retract, or halt the surgical scope longitudinally relative to the body wall, or
(B) provide a notification to a user of the system.

37. The system of claim 36, further comprising a sheath configured to be inserted through the surgical opening and slidably receive the surgical scope, wherein the feed device is operable to selectively advance and retract the surgical scope and the sheath collectively relative to the body wall.

38. The system of claim 36, wherein the optical distance measurement system includes at least one of a sensor or a light emitter arranged at the distal tip of the surgical scope.

39. A method of operating a system that includes a surgical scope having a distal tip with a lens configured to visualize an anatomical structure within a body cavity of a patient, a feed device, and a controller, the method comprising:
(a) controlling the feed device with the controller to advance the surgical scope distally relative to a body wall of the patient such that the distal tip advances toward the anatomical structure;
(b) measuring a present target distance from the distal tip to the anatomical structure;
(c) comparing the present target distance to a threshold target distance with the controller; and
(d) based on the comparison, controlling the feed device with the controller to at least one of:
(i) advance, retract, or halt the surgical scope longitudinally relative to the body wall, or
(ii) provide a notification to a user of the system.

40. The method of claim 39, further comprising, when the present target distance is greater than the threshold target distance by a difference, controlling the feed device with the controller to advance the surgical scope distally relative to the body wall by a distance equal to the difference.
